(19) United States
(12) Patent Application Publication
(10) Pub. No.: US 2017/0056573 A1
(43) Pub. Date: Mar. 2, 2017

(54) Lacteal Extractor Safety System and Method for Pump System

(71) Applicant: MEDELA HOLDING AG, Baar (CH)

(72) Inventor: Raymond Holtz, Chicago, IL (US)

(21) Appl. No.: 15/249,768

(22) Filed: Aug. 29, 2016

Related U.S. Application Data
(60) Provisional application No. 62/210,841, filed on Aug. 27, 2015.

(51) Int. Cl. A61M 1/06; G05D 16/06; A61M 39/22; F16K 51/02

(52) U.S. Cl. CPC A61M 1/062; F16K 51/02; G05D 16/06; A61M 1/066; A61M 1/06; A61M 39/22

(57) ABSTRACT

A mechanical over-vacuum safety system and method of use for a vacuum pump system designed to extract milk from lactating mothers. The over-vacuum safety system includes at least one mechanical safety valve configured to reduce a vacuum in the pump system given an over-vacuum condition. The mechanical safety valve(s) may be automatically actuated in response to specific vacuum ranges, such as an over-vacuum conditions ranging between negative 350 mmHG to negative 300 mmHG. The mechanical safety valve may allow fluid flow by folding.

### CROSS-REFERENCE TO RELATED APPLICATION

This application claims the benefit of the filing date of U.S. Provisional Patent Application No. 62/210,841 filed Aug. 27, 2015 and entitled “Lacteal Extractor Safety System and Method for Pump System,” the entire disclosure of which is hereby expressly incorporated by reference herein.

### TECHNICAL FIELD

The present disclosure relates generally to a mechanical safety system and method for a pump system. More particularly, the disclosure relates to a mechanical over-vacuum safety system and method for a vacuum pump system designed to extract milk from lactating mothers.

### BRIEF DESCRIPTION OF THE DRAWINGS

FIG. 1 is a side view of an exemplary embodiment constructed in accordance with the principles herein;

FIG. 1A is an side view of an exemplary mechanical safety valve system constructed in accordance with the principles herein;

FIG. 2 is a side view, partially in section, of another exemplary embodiment constructed in accordance with the principles herein;

FIGS. 3a-b are sectional views taken along lines 3A of FIG. 2 with the valve in a unsealed condition and sealed condition, respectively;

FIG. 3c is an exemplary embodiment of a portion of a fluid flow member configuration that includes at least one component that prevents the valve from fully entering the flow unit given an over vacuum condition in a pump system;

FIGS. 3d and 3e are pictures of a valve sealing the flow unit of the system under vacuum, and automatically venting under an over vacuum condition of the system, respectively.

FIG. 4 is a flow chart of an exemplary method of constructing a lacteal extractor safety system in accordance with the principles herein.

FIG. 5 is a front perspective view of an embodiment of a valve sealing the flow unit of the system under a vacuum and automatically venting under an over vacuum condition of the system.

FIG. 6 is atop view of the valve illustrated in FIG. 5.

FIG. 7 is a side view of the valve illustrated in FIGS. 5 and 6.

FIG. 8 is a bottom view of the valve illustrated in FIGS. 5-7.

FIG. 9 is a cross-sectional view of the valve illustrated in FIGS. 5-8 taken along line 9-9 in FIG. 6.

Throughout the various figures, like reference numbers refer to like elements of the exemplary system components. Elements can be combined in all possible combinations that can be formed to achieve exemplary systems not specifically set forth in the figures, in accordance with the principles herein.

### DETAILED DESCRIPTION

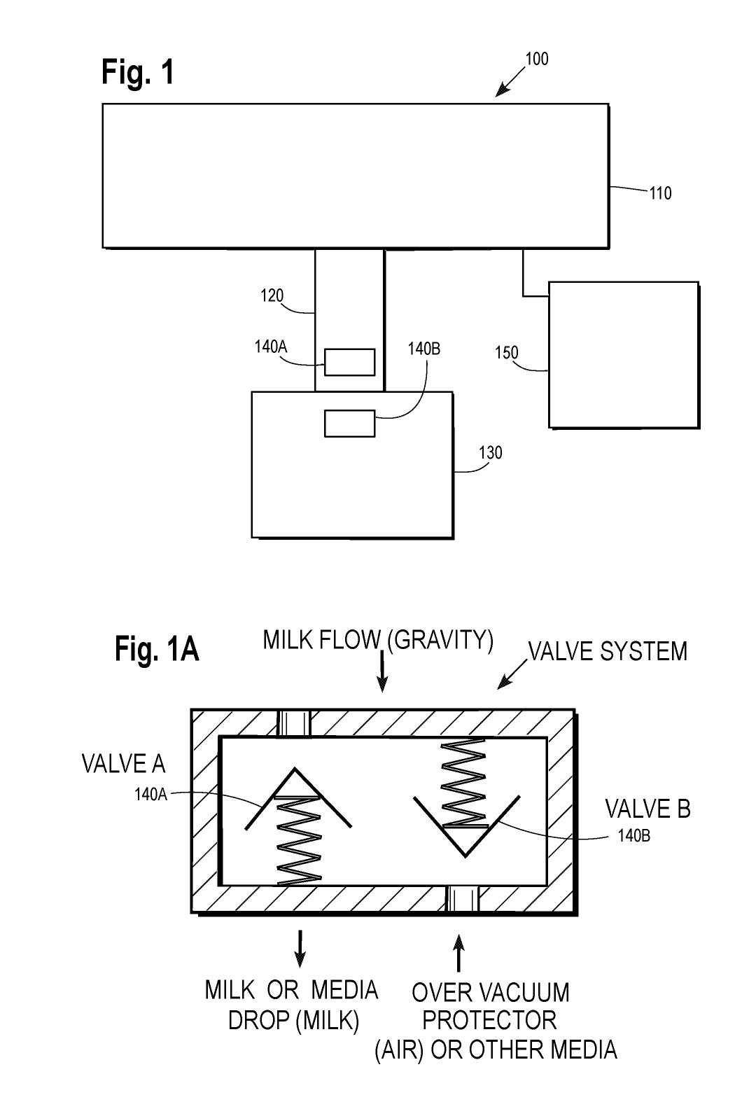

FIG. 1 illustrates an exemplary vacuum pump system shown generally at 100, constructed in accordance with the principles herein. The pump system 100 includes a lacteal extractor 110 connectable to a lactating mother, and a fluid flow member 120 through which milk extracted from the mother can flow. The fluid flow member 120 can be formed of any length, shape, and material, and can provide or contain one or more components designed to convey milk from the lacteal extractor 110 of the pump system 100 to a collection container 130. A vacuum generating system 150 can be either directly or indirectly connected to the lacteal extractor 110.

In accordance with the principles herein, vacuum generated by the vacuum generating system 150 and delivered to the lacteal extractor 110 can advantageously be automatically mechanically regulated to reduce the chance of an over vacuum condition in the system 100. To this end, one or more mechanical valves can be positioned between the milk collection container 130 and the lacteal extractor 110, where the one or more mechanical valves are designed to provide a mechanical safety valve system that is automatically actuated in response to specific vacuum ranges that can cause an over vacuum condition in the system. Although numerous pump systems employ mechanical valves to facilitate milk flow from a lacteal extractor to a container, the flow valves of conventional systems do not function as mechanical safety valves within the fluid flow path, and are not configured and tuned to mechanically open when subjected to an over vacuum condition of the system.

Alternately, the mechanical safety valve system could be positioned outside of the fluid flow path of the system, such as, for example, in the vacuum line or anywhere else in the system. In certain circumstances, the mechanical safety valve system can be positioned to serve as both a safety valve and a milk extraction valve, as illustrated according to the exemplary embodiments herein.

In an exemplary embodiment constructed in accordance with the principles herein, a safety valve 140A can be positioned within the flow unit 120 of the system 100. In yet another exemplary embodiment, a safety valve 140 B can be positioned within the collection container 130 of the system 100. In still another exemplary embodiment depicted in FIG. 1A, a mechanical safety valve system of more than one safety valve, such as a safety valve 140A and a safety valve 140B, or any number of valves that form a suitable safety valve system, can be position in the system 100 downstream from the lacteal extractor 110 to form the mechanical safety valve system. Suitable mechanical safety valve systems constructed in accordance with the principles herein automatically and mechanically reduce vacuum in the system by altering its geometry to allow communication with atmospheric pressure under certain vacuum ranges.

Typical vacuum ranges in which an over vacuum condition can occur in the system can include, for example, from around negative 300 mmHg to about negative 350 mmHg. If desired, the vacuum ranges in which an over vacuum condition can occur can be fine-tuned with feedback from a mother using the system based her comfort levels. To this end, an adjustment, mechanical, electrical or both, of vacuum ranges can be made in the system based on the feedback from the mom. Such adjustment will not affect the efficacy of the valve, but can serve to reduce the pressure at the upper limit of the over vacuum condition, rendering operation of the device more comfortable for the mom.

In order for the mechanical safety valve system to function automatically in the system one must employ the right combination of material thickness of the valve, material properties of the valve, open or flow through diameter of the valve, and the beam or bend length of surface of the valve that achieves the safety feature in relation to the applied pressure on the valve during operation.

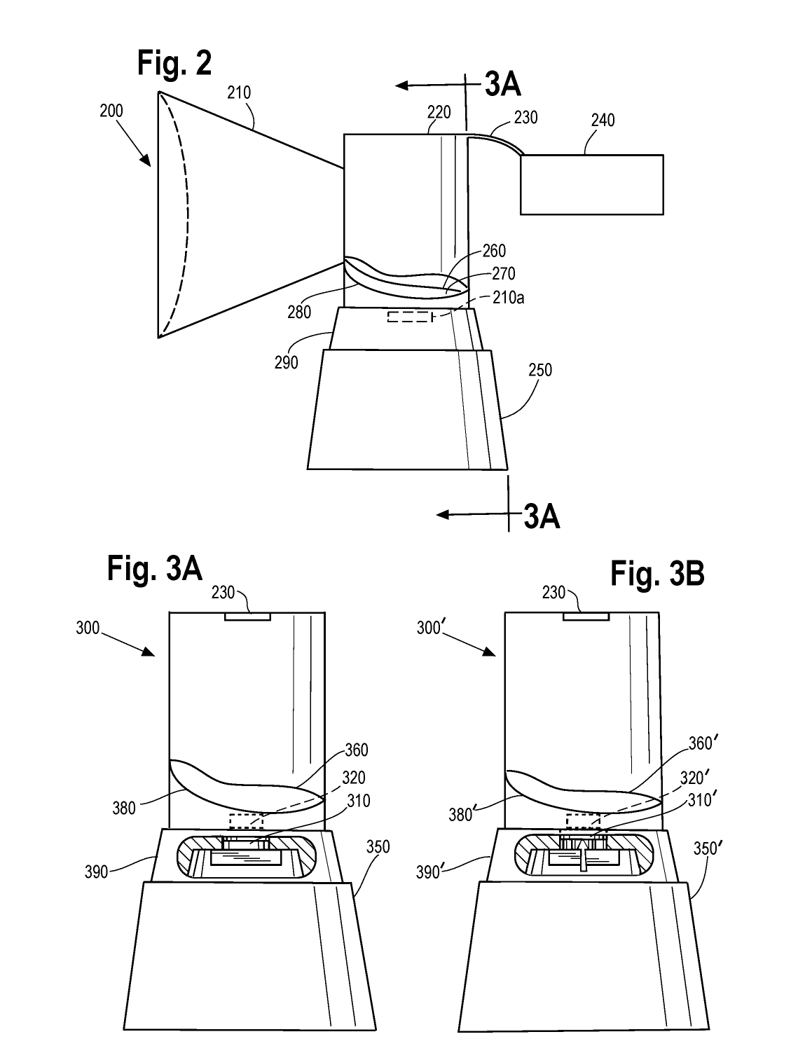

As illustrated in FIG. 2, another exemplary embodiment of a system shown generally at 200 and constructed in accordance with the principles herein can include an exemplary lacteal extractor formed of a breastshield 210, an adapter 220 connected via tubing 230 to a vacuum pump 240 and a collection container 250. A separator unit 260 can be included to separate milk, or other fluid media, flowing from the breastshield 210 from entering the tubing 230 or the vacuum pump 240. An opening 270 can be provided along a bottom interior wall 280 of the adapter 220. The bottom interior wall 280 can be provided in the system 200 to minimize the work required by the vacuum generating system 240 to achieve desired vacuum ranges in accordance with a program or pattern of the pump system 200. The pump system 200 can be designed so that the program or pattern is selectable by a mother.

The system 200 can further include a valve system 290 containing a flexible, movable valve 210a. The valve system 290 is connectable to the adapter 220 and the container 230. Known lacteal extraction systems include a flow valve to the container. Some known systems include a relief or safety valve, but not an automatic mechanical safety valve within the system. For example, a relief valve can be connected with a breastshield as discussed in U.S. Pat. No. 8,070,716 to Sutrina et al. Thus, in accordance with the principles herein the exemplary valve 210a can serve, advantageously, as both a fluid flow control valve and as an automatic mechanical safety valve system within the fluid flow.

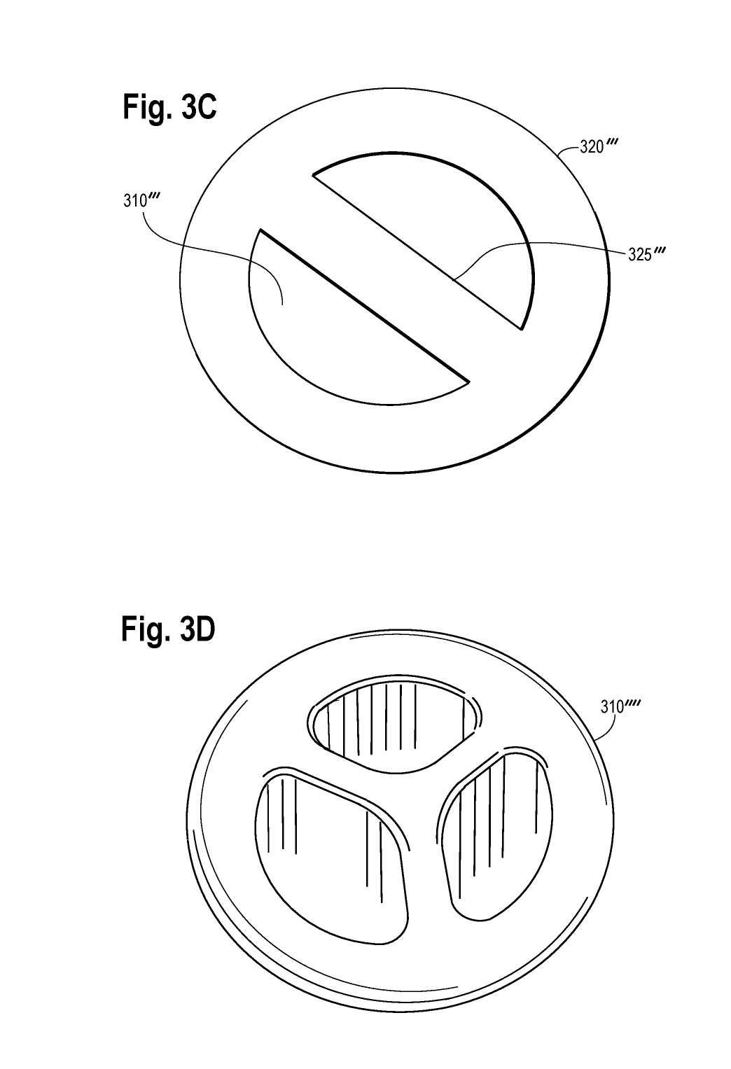

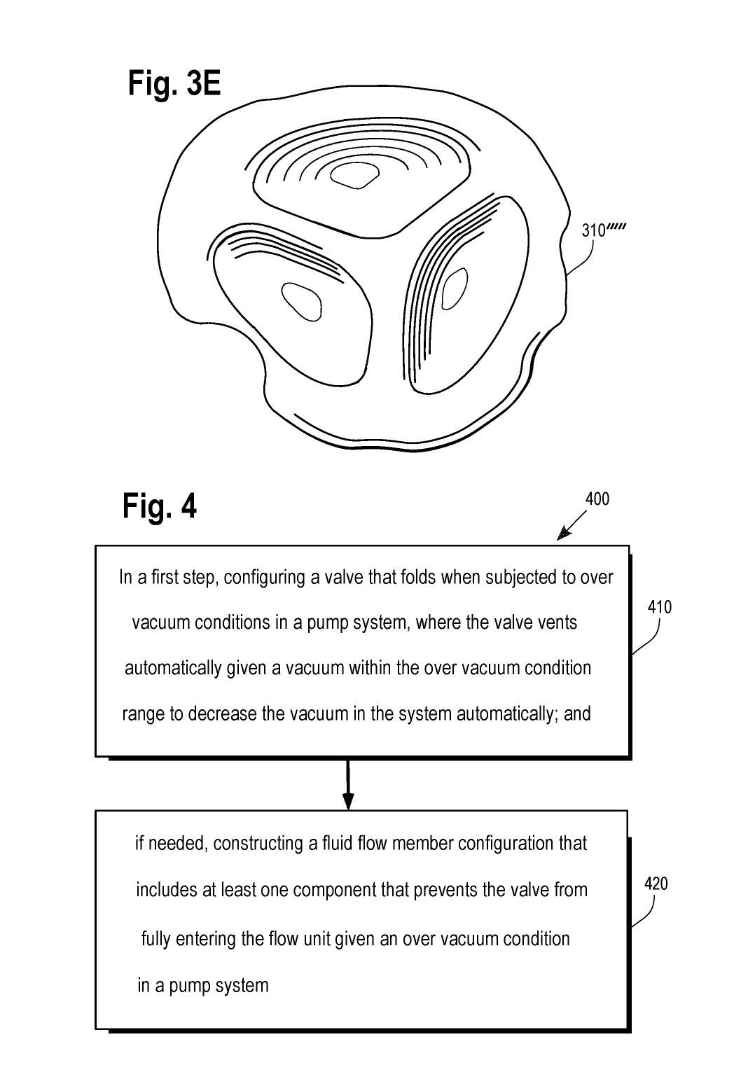

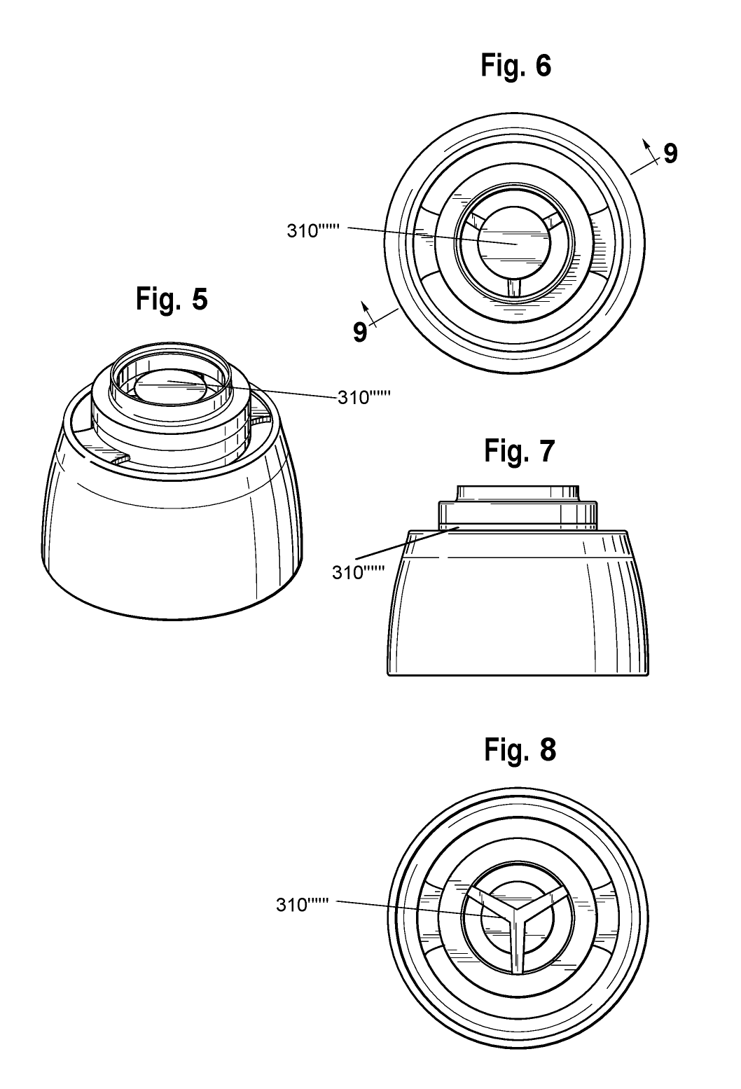

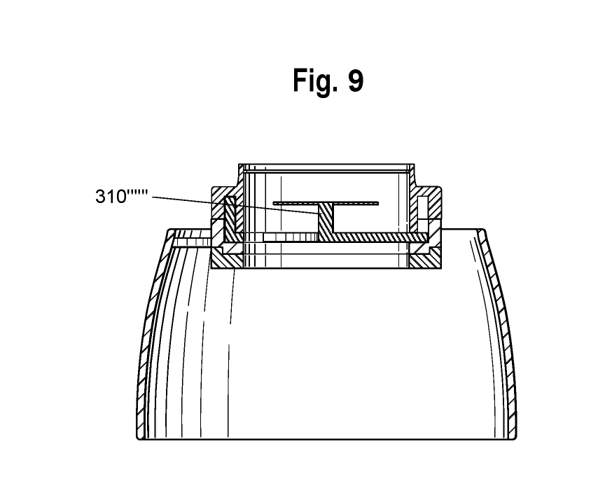

As illustrated in FIGS. 3a to 3e, an exemplary mechanical safety valve system can include a flexible, movable valve 310 in a valve system 390. The valve 310 can control fluid flow in the system, and can allow fluid to pass into a collection container 350. The valve 310 can also serve as a mechanical safety valve system when the valve is constructed such that it limits the volume of the system in order to achieve upper vacuum levels, or negative pressure values, and alters the geometry of the valve to reduce vacuum levels when an over vacuum condition occurs in the system

In an exemplary embodiment shown in FIG. 3b, a valve 310′ can seal an aperture 320′ leading to a container in the system when vacuum is applied. In this sense, the valve 310′ can function similarly to conventional lacteal extraction flow control valves.

In the exemplary embodiment shown in FIG. 3c, an aperture or opening 320′ within a wall of a flow unit can include one or more ribs 325′″, designed to prevent a valve 310′″ from being pulled into the flow unit when an over vacuum condition occurs. In this embodiment the valve 310′″ is sufficiently flexible under high vacuum conditions that the valve can flex and fold when sufficient vacuum is applied. As a result, ribs 325′″ or other similar structure(s), such as a mesh or any other alternate structure(s), ensure that the valve remains in a position within the system to limit the system volume under normal vacuum conditions, and to alter the geometry of the valve in the system under over vacuum conditions.

Moreover, in the embodiment of FIGS. 3d and 3e, the system is configured such that the valve 310″″, 310′″″ respectively can actually fold or bend as indicated to reduce vacuum of the system mechanically and automatically when an over vacuum condition occurs during operation. The valve can be designed to have physical characteristics and shape such that a portion of the valve can undergo stretching in an over vacuum condition that results in the bending or folding of the valve 310 as shown in FIG. 3e. In the exemplary embodiments of FIGS. 3d and 3e, the aperture of the fluid unit includes three ribs that ensure the valve remains in a position within the system to limit the system volume under normal vacuum conditions, and to alter the geometry of the valve in the system under over vacuum conditions.

Although any suitable method can be employed in accordance with a method of the present disclosure, it is contemplated that particular advantage can be achieved through the use of manufacturing methods such as, for example an exemplary method of forming a mechanical safety valve system for a pump system illustrated in FIG. 4. A method in accordance with the principles herein, shown generally at 400 can include the step at 410 of configuring a valve that folds when subjected to over vacuum conditions in a pump system, where the valve vents automatically given a vacuum within the over vacuum condition range to decrease the vacuum in the system automatically; and at step 420, if needed, constructing a fluid flow member configuration that includes at least one component that prevents the valve from fully entering the flow unit given an over vacuum condition in a pump system.

While exemplary embodiments of the present disclosure are provided herein, various changes and modifications can be made without departing from the spirit and scope of the invention. The scope of the invention is indicated in the appended claims, and all changes that come within the meaning and range of equivalents are intended to be embraced therein. For example, variations in the forming and/or any other features described in the present disclosure are within the spirit and meaning of the appended claims.

What is claimed is:

1. A pump system for lacteal extraction comprising:
a mechanical safety valve within a flow path of the system, the safety valve configured to reduce a vacuum in the pump system given an over-vacuum condition.

2. The pump system of claim 1, further comprising:
a lacteal extractor;
a fluid flow member fluidly connected to the lacteal extractor;
a collection container connected to the fluid flow member;
wherein the mechanical safety valve is positioned between the lacteal extractor and the collection container.

3. The pump system of claim 1, further comprising:
a lacteal extractor;
a fluid flow member fluidly connected to the lacteal extractor;
a collection container connected to the fluid flow member;
wherein the mechanical safety valve is positioned within the collection container.

4. The pump system of claim 1, wherein the pump system includes two or more mechanical safety valves located downstream of a lacteal extractor.

5. The pump system of claim 1, further comprising a lacteal extractor that is a breastshield.

6. The pump system of claim 1 further comprising:
an adapter connected by tubing to a vacuum pump and a collection container, wherein the mechanical safety valve is in the adapter.

7. The pump system of claim 6, wherein the adapter comprises an opening on a bottom wall of an adapter, and the mechanical safety valve is at the opening .

8. The pump system of claim 7, wherein the opening is partially covered by ribs, mesh, or an alternate structure.

9. The pump system of claim 7, wherein the adapter includes a separator unit for milk.

10. The pump system of claim 1, wherein the over-vacuum condition occurs between negative 300 mmHG and negative 350 mmHG.

11. The pump system of claim 1, wherein the mechanical safety valve is flexible.

12. The pump system of claim 1, wherein the mechanical safety valve undergoes stretching or folding in an over-vacuum condition.

13. A method of forming a mechanical safety valve system for a pump system including:
configuring a valve that allows fluid flow when subjected to over vacuum conditions in a pump system , wherein the valve vents automatically given a vacuum within the over-vacuum condition range to decrease the vacuum in the system automatically.

14. The method of claim 13, and constructing a fluid flow member configuration that includes at least one component that prevents the valve from fully entering a flow unit of the pump system given an over-vacuum condition in a pump system.

15. The method of claim 13, wherein the over-vacuum condition range is negative 350 mmHG to negative 300 mmHG.

16. The method of claim 13, wherein the valve allows fluid flow by folding.

17. The method of claim 14, wherein the component that prevents the valve from fully entering the flow unit is ribs, mesh, or an alternate structure.

18. The method of claim 13, wherein the pump system includes an adapter and the valve is in the adapter.

19. The method of claim 18, wherein the adapter has an opening on a bottom wall, and the component prevents the valve from fully traveling through the opening.

20. The method of claim 18, wherein the adapter includes a separator unit for milk.

21. A pump system for lacteal extraction comprising:
one or more mechanical valves positioned within a collection container that are designed to provide a mechanical safety valve system that is automatically actuated in response to specific vacuum ranges that can cause an over vacuum condition in the system.

22. The pump system of claim 21, where the pump system comprises a lacteal extractor directly or indirectly connected to a vacuum generating system.

23. A pump system for lacteal extraction comprising:
one or more mechanical valves positioned within a flow unit that are designed to provide a mechanical safety valve system that is automatically actuated in response to specific vacuum ranges that can cause an over vacuum condition in the system.

24. The pump system of claim 23, where the pump system comprises a lacteal extractor directly or indirectly connected to a vacuum generating system .
